(19) United States
(12) Patent Application Publication
(10) Pub. No.: US 2004/0031861 A1
(43) Pub. Date: Feb. 19, 2004

(54) Essential oils in additive concentrates

(76) Inventor: Rod A. Garcia, Montgomery, TX (US)

Correspondence Address: Kurt S. Myers, 7634 Braesdale, Houston, TX, 77071, US

(21) Appl. No.: 10/412,403

(22) Filed: Apr. 12, 2003

Related U.S. Application Data
(60) Provisional application No. 60/404,049, filed on Aug. 16, 2002.

(51) Int. Cl. A61K 7/46

(52) U.S. Cl. 239/34

(57) ABSTRACT

The present invention is directed to a free flowing solid concentrate containing essential oils and methods for forming the concentrates.

### RELATED APPLICATION

This application is based on provisional application Serial No. 60/404,049, filed Aug. 16, 2002.

### FIELD OF THE INVENTION

The present invention is directed to the incorporation of essential oils in additive concentrates. The essential oil additive concentrates of the present invention are free flowing solids that are used to produce a variety of products.

### BACKGROUND OF THE INVENTION

U.S. Pat. No. 3,244,586 discloses o-pyridyl phosphates and phosphorothioates as pesticide compounds. These compounds are incorporated into films and are used presently in banana bags using the commercially available product Dursban® insecticide, trademark of Dow Chemical Company.

U.S. Pat. No. 3,567,119 discloses methods for the incorporating of fragrance compounds or oil bouquets and/or topical antifungal or antibacterial agents, insect repellant compounds and certain odoriferous medicaments into polymeric or natural materials so that the fabricated product possesses the properties imparted by the additive or additives for a long period of time.

U.S. Pat. No. 3,655,129 discloses methods of making cast films that contain various volatilizable liquids that permeate slowly through and out of the films.

U.S. Pat. No. 3,685,734 discloses an assembly of a smooth-surfaced layer of soft vinyl plastisol containing a fragrance or other volatile organic substance coated on a base and protectively covered with a smooth flexible sheet having low vapor transmission.

U.S. Pat. No. 4,159,314 discloses an insect repellant animal collar wherein a strip of flexible polymeric material adapted to be worn on the neck of the animal is impregnated with a thujane derivative.

U.S. Pat. No. 4,320,112 discloses a receptacle such as a plastic trash can or bag that contains an insect and animal repellent for ridding areas containing the receptacles of annoying insects and for preventing animals from turning over or destroying the receptacles when full.

U.S. Pat. No. 4,871,541 discloses a process involving forming a clathrate compound of fenitrothion with cyclodextrin or a starch decomposition product containing cyclodextrin and granulating into a dry powder, melt-mixing the dry powder with a synthetic material and molding the mixture into pellets. The pellets are molded in the form of film by calendaring or by extrusion molding.

U.S. Pat. No. 4,888,174 discloses a method of retarding the rate of migration of O-halopyridl phosphate insecticide from polyethylene articles.

U.S. Pat. No. 5,246,919 discloses a fragrant material that is comprised of an aggregate of small sodium chloride granules and having a fragrant oil and optionally a water soluble dye dispersed in the material.

U.S. Pat. No. 5,688,509 discloses a controlled-release insect repellent device that comprises an insect repellent composition contacting a substrate. The repellent compound may be chosen from the group consisting of essential oils and active ingredients of essential oils.

U.S. Pat. No. 5,885,600 discloses a composition that has insect repellent properties made from cold processed extracted oils and an antioxidant, particularly from a combination of Neem, Citronella and Cedarwood oils.

U.S. Pat. No. 6,270,753 discloses a fragrance-containing composition for paper substrates consists essentially of a polyvinyl chloride (PVC) plastisol and one or more fragrances, for example, an insect repellent.

U.S. Pat. No. 6,346,297 discloses a method of coating a paper substrate with a composition that consists essentially of a polyvinyl chloride (PVC) plastisol and one or more fragrances, for example, an insect repellent.

### SUMMARY OF THE INVENTION

The present invention is directed to free flowing concentrates that comprise a carrier and at least one essential oil. These concentrates may be used as additives in the manufacture of films or molded products,

### BRIEF DESCRIPTION OF THE EMBODIMENTS OF THE INVENTION

The unique products of the present invention are directed to one or more essential oil incorporated with a carrier into a free flowing solid concentrate. An “essential oil”, as used herein, is any essential oil including any of the more than thirty commercially available essential oils such as almond oil, anise oil, bay oil, caraway oil, cardamon oil, cedar oil, celery oil, chamomile oil, cinnamon oil, citronella oil, clove oil, coriander oil, corn oil, cottonseed oil, cumin oil, dill oil, eucalyptus oil, fennel oil, garlic oil, geranium oil, ginger oil, grapefruit oil, lemon oil, lime oil, linseed oil, mint oil, parsley oil, pepper oil, peppermint oil, rose oil, rosemary oil, sesame oil, soybean oil, spearmint oil, orange oil, thyme oil, turmeric oil, wintergreen oil (oil of wintergreen) or any of the natural or synthetic active ingredients in essential oils such as citronellal, methyl salicylate, ethyl salicylate, propyl salicylate, citronellal, safrole and D-limonene.

The liquid essential oils are compounded with a carrier that absorbs the oil or permit's the incorporation of the essential oil into the carrier to produce a free flowing solid concentrate. The solid free flowing essential oil concentrate may be used as a product or more specifically is added with plastic resins before or in the plastic-forming process to incorporate the essential oil into the finished product. Specific examples of finished products are plastic films or extruded plastic products, such as food trays or clothe hangers. The solid free flowing concentrate and the plastic resin are introduced into an extruder, fully dispersing the essential oil as an additive concentrate in the plastic resin, to produce the products of the present invention. Plastic films, either blown films or cast films (depending on the specific film resins used, the equipment used and the end-use of the plastic film); plastic molded products, either injection molded or blow molded (depending on the specific plastic resins used, the equipment used and the end-use of the plastic molded product) are products that are manufactured or produced using the essential oil additive concentrates of the present invention.

To illustrate the variety of production methods to produce the essential oil concentrates of the present invention, the following examples are given:

### EXAMPLE 1

| | 1A | 1B | 1C |
|---|---|---|---|
| MP300* | 40 grams | 30 | 20 |
| Cedar oil | 60 grams | 70 | 80 |

*MP300 is a micro porous LLDPE pellet from Akzo Nobel

This example illustrates that the essential oil, cedar oil, is blended with the carrier in a mixer and that concentrations of up to 80 wt % oil in the carrier produced a free flowing solid concentrate. A similar result is obtained using other essential oils. Other porous resins are commercially available, such as porous polypropylene, EVA and HDPE or LDPE to mention a few.

### EXAMPLE 2

| | 2A | 2B | 2C |
|---|---|---|---|
| Cedar oil | 10 grams | 30 | 50 |
| 9820* | 90 grams | 70 | 50 |

*9820 is a polyethylene of Union Carbide

The polyethylene and cedar oil are blended in a Henchel or high intensity mixer and then compounded through a twin-screw compounder (extruder). Instead of using a high intensity mixer, the oil can be injected down steam on the twin-screw extruder after the resin is melted to be fully mixed or incorporated into the resin. The essential oil concentrate produced is either cut under water or strand cut in pellets. The pellets are free flowing solids containing up to 50 wt % essential oil.

This example illustrates that a free flowing solid pellet is produced by compounding in an extruder. Other resins such as low density polyethylene (LDPE), EVA, polypropylene, polystyrene and nylon are used in place of the LLDPE to produce the essential oil concentrates of the present invention. Other essential oils such as wintergreen, D-limonene, eucalyptus, garlic and peppermint are used in place of the cedar oil.

### EXAMPLE 3

| | 3A | 3B | 3C |
|---|---|---|---|
| Cedar Oil | 50 grams | 60 | 80 |
| Hubersorb 600* | 50 grams | 40 | 20 |
| | 3X | 3Y | 3Z |
| Cedar Oil | 50 grams | 60 | 80 |
| Micro-cel** | 50 grams | 40 | 20 |

*Hubersol 600 is a silica product of J. M. Huber Corp. and **Micro-cel is a silica product of Johns Manville

This example illustrates that a free flowing solid concentrate is produced by simple mixing with a high surface area silica material. These concentrates are compatible with the plastic resins to form finished products containing the essential oil.

### EXAMPLE 4

In a pellet mill (California pellet mill type) is placed 35 gr. Cedar oil, 45 gr. of film grade polyethylene, and 20 gr. polyethylene wax to produce a free flowing pellet This example illustrates that a free flowing solid pellet is produced by compounding in a pellet mill using wax to form a concentrate for processing film at low temperature. Steric acid may replace all or some of the wax as a binder or carrier.

The following examples illustrate the use of the concentrates of the present invention in making film products.

### EXAMPLE 5

Film samples are prepared as follows:

Samples A, B, & C were produced from 50 wt % cedar oil concentrate

Sample D was produced from a 70 wt % cedar oil concentrate

Sample E was produced from a 60 wt % cedar oil concentrate

Concentrate resin was LLDPE micro porous polyethylene, a product of Akzo Nobel

| | 5A | 5B | 5C | 5D | 5E |
|---|---|---|---|---|---|
| LDPE* | 2.8 pound | 2.8 | 2.8 | 2.8 | 0 |
| 4903** | 1.2 pound | 1.2 | 1.2 | 1.2 | 4.0 |
| Cedar conc. | 5 gram | 10 | 20 | 50 | 70 |
| % oil in film | 0.19 | 0.37 | 0.74 | 1.9 | 2.6 |

*film grade LDPE of Dow Chemical Company;
**MDPE of Phillips Chemical Company.

These samples are run in a film blowing apparatus making film at approximately 1 mil thickness.

Using the master batch technique illustrated above, film samples are produced with concentrates of various concentrations of cedar oil. Film is produced at three levels of cedar oil: 1.5 wt %, 3.0 wt % and 4.0 wt %. The film is made into banana bags, tubular with both ends open and having a plurality of openings in the film. The bags are placed over the banana bunch on the tree to repel insects and protect the bananas from birds, bats and the wind while still green.

### Example 6

| | 6A | 6B | 6C | 6D |
|---|---|---|---|---|
| 4903* | 1.2 pound | 1.2 | 1.2 | 1.2 |
| 1539** | 2.8 pound | 2.8 | 2.8 | 2.8 |
| M B*** | 120 grams | 120 | 60 | 60 |
| Green color | | 60 | | 30 |
| | | grams | | |

*4903 is a LDPE of the Phillips Chemical Company; **1539 is a LLDPE of Union Carbide Company; and ***is a Master Batch of 35 wt % cedar oil, 50 wt % LDPE and 15% Mirocel E Silica, a product of Johns Mansville.

The foregoing examples are repeated with the following master batch or concentrates using styrene modified polymers as the carrier to make the free flowing concentrate:

| 6E | | 6F | | 6G | |
|---|---|---|---|---|---|
| Vector 4113 | 70 wt % | Vector 4211 | 60 wt % | Vector 6030-D | 60 wt % |
| Cedar Oil | 30 wt % | Wintergreen | 40 wt % | D-limonene | 40 wt % |

Vector 4113 (SIS/SI) is a linear styrene-isoprene-styrene triblock/styrene-isoprene diblock copolymer

Vector 4211 (SIS) is a styrene-isoprene-styrene linear triblock copolymer

Vector 6030-D (SBS) is a styrene-butadiene block copolymer

| 6H | | 6J | | 6K | |
|---|---|---|---|---|---|
| Vector 2518 | 70 wt % | KR03 | 40 wt % | G1650 | 80 wt % |
| Eucalyptus | 30 wt % | Cedar | 60 wt % | Garlic | 20 wt % |

Vector 2518 (SBS) is a styrene-butadiene-styrene triblock copolymer

KRO3 is a styrene-butadiene copolymer (K-resin) from Phillips

G1650 (SEBS) is a styrene-ethylene-butylene block copolymer from Shell

6M

| | |
|---|---|
| G1701X | 40 wt % |
| Peppermint | 60 wt % |

G1701X (SEP) is a styrene-ethylene-propylene block copolymer

These samples are run in a film blowing apparatus with the film resin mixed with the free flowing concentrate in the extruder making film at approximately 1 mil thickness.

The foregoing examples illustrate the production of a free flowing solid concentrate of one essential oil and incorporated with or dispersed in a film forming resin before or in the film-forming process to produce a plastic film containing the essential oil. However, more than one essential oil may be incorporated into the film. The essential oils are not equivalent in the repelling of a particular insect, one essential oil may have more power on certain insects than another oil. Also the effect on the product being protected is different with different essential oils. The following examples illustrate that more than one essential oil may be incorporated in the concentrate and then dispersed within a film.

### EXAMPLE 7

Sample A cedar oil+wintergreen oil+resin+green color

Sample B cedar oil+D-limonene oil+resin+green color

Sample C cedar oil+eucalyptus oil+resin+green color

Sample D wintergreen oil+D-limonene oil+resin+green color

Sample E wintergreen oil+eucalyptus oil+resin+green color

Sample F D-limonene oil+eucalyptus oil+resin+green color

| | 7A | 7B | 7C | 7D | 7E | 7F |
|---|---|---|---|---|---|---|
| 4903* | 1.2 pounds | 1.2 | 1.2 | 1.2 | 1.2 | 1.2 |
| 1539** | 2.8 pounds | 2.8 | 2.8 | 2.8 | 2.8 | 2.8 |
| Cedar*** | 30 grams | 30 | 30 | | | |
| Wg*** | 30 grams | | | 30 | 30 | |
| D-limonene*** | | 30 grams | | 30 | | 30 |
| Eucalyptus*** | | | 30 | | 30 | 30 |
| | | | grams | | | |
| Green Color | 25 grams | 25 | 25 | 25 | 25 | 25 |

*4903 LDPE of Philips Chemical Co.;
**1539 LLDPE of Union Carbide Co.;
***a concentrate of 60 wt % oil and 40 wt % porous PE resin

Each of the above samples is blown into green film. This example illustrates that more than one essential oil concentrate may be used to make film. Films with more than one essential oil will have different degrees of freshness and odor repelling characteristics. The film may be run through bag making apparatus to make bags for fruit such as grapes or used to keep products such as cut flowers, insect free.

### EXAMPLE 8

This examples illustrates that as many as four different essential oils may be incorporated into a concentrate and then into a film.

| | 8A | 8B | 8C | 8D |
|---|---|---|---|---|
| 1539* | 4.0 pounds | 4.0 | 4.0 | 4.0 |
| Cedar** | 30 grams | 30 | 0 | 30 |
| D-limonene** | 30 | 0 | 30 | 30 |
| Eucalyptus** | 30 | 30 | 30 | 30 |
| Wintergreen** | 0 | 30 | 30 | 30 |
| Green color | 25 grams | 25 | 25 | 25 |

*1539 LLDPE of Union Carbide; **a concentrate of 50 wt % oil and either MP300 or styrene modified polymer.

### EXAMPLE 9

This example illustrates still another group of carriers that are used in producing the essential oil concentrates of the present invention. These carriers are selected from the natural and synthetic zeolites or molecular seive materials.

| | 9A | 9B |
|---|---|---|
| Zeodex 110-40* | 50 wt % | 40 wt % |
| Essential oil** | 50 wt % | 60 wt % |

*a natural zeolite from Zeodex International
**an essential oil selected from the group of cedar oil, wintergreen, D-limonene, eucalyptus, garlic and peppermint.

| | 9C | 9D |
|---|---|---|
| Abscents 300* | 50 wt % | 40 wt % |
| Essential oil** | 50 wt % | 60 wt % |

*a synthetic zeolite from UOP
**an essential oil selected from the group of cedar oil, wintergreen, D-limonene, eucalyptus, garlic and peppermint.

The foregoing examples are used for illustration of the present invention. The examples illustrate that essential oils may be substituted for one another or more than one essential oil may be used. The examples illustrate that the carrier material may be selected from a group of materials including porous resins (see Example 1), compounded resins (see Example 2), silica compounds (see Example 3), wax (see Example 4), styrene modified polymers (see Example 6), and natural or synthetic zeolites (see Example 9).

1. A free flowing concentrate for compounding with plastic resin comprising:
1 wt. % to 80 wt. % of an essential oil; and
1 wt. % to 50 wt. % of a carrier.

2. A concentrate according to claim 1 wherein said essential oil is selected from the group consisting of cedar oil, wintergreen, D-limonene, eucalyptus, garlic and peppermint.

3. A concentrate according to claim 1 wherein there is more than one essential oil.

4. A concentrate according to claim 1 wherein said carrier is a micro-porous resin.

5. A concentrate according to claim 1 wherein said carrier is selected from the group of high structure absorbents consisting of silica, minerals and clays.

6. A concentrate according to claim 1 wherein said high surface area carrier is silica.

7. A concentrate according to claim 1 wherein said carrier is a styrene modified polymer.

8. A concentrate according to claim 1 wherein said carrier is a natural or synthetic zeolite.

9. A concentrate for compounding with plastic resin comprising:
a blend of at least two essential oils and a carrier for producing a free flowing solid plastic additive.

10. A concentrate according to claim 9 wherein said blend includes wintergreen and cedar oil.

11. A method for producing a free flowing concentrate of essential oil comprising:
blending in a high intensity mixer a high surface area carrier with up to 50 wt. % of an essential oil.

12. A method for producing a free flowing concentrate of essential oil comprising:
blending in a high intensity mixer a micro porous plastic resin with up to 50 wt. % of an essential oil.

13. A method for producing a free flowing concentrate of essential oil comprising:
mixing a high surface area structure silica, mineral or clay with up to 80 wt. % of an essential oil.

14. A method for producing a free flowing concentrate of essential oil comprising:
mixing in a pellet mill a film forming resin and an essential oil in the presence of a homogenizing agent.
